(19) United States
(12) Patent Application Publication
(10) Pub. No.: US 2020/0000619 A1
(43) Pub. Date: Jan. 2, 2020

(54) Performance Knee Orthosis

(71) Applicants: Robert Myron Schipman, Lincoln, NE (US); Scott Edwin Strasburger, Lincoln, NE (US); Doak Matthew Ostergard, Lincoln, NE (US)

(72) Inventors: Robert Myron Schipman, Lincoln, NE (US); Scott Edwin Strasburger, Lincoln, NE (US); Doak Matthew Ostergard, Lincoln, NE (US)

(21) Appl. No.: 16/455,547

(22) Filed: Jun. 27, 2019

Related U.S. Application Data
(60) Provisional application No. 62/691,040, filed on Jun. 28, 2018.

(51) Int. Cl. A61F 5/01

(52) U.S. Cl. CPC A61F 5/0123; A61F 2005/0137; A61F 2005/0146

(57) ABSTRACT

The embodiments to the disclosure relate generally to the field of orthopedic braces, and more particularly orthopedic braces made specifically for the knee, that provide prophylactic, post-injury, or postoperative support needed to stabilize the associated joints, tendons, and ligaments, while simultaneously allowing for the required flexion and extension and internal and external rotation of the knee, mimicking the joint's true physiologic motion.

### BACKGROUND OF THE INVENTION

We often take for granted how easily our bodies move. Movement often occurs without thought. However, when we understand how simple movements require complex interactions between various bones, ligaments, and muscles, we realize a complex system is at work.

The knee is the largest joint in the human body and is formed by the articulation of the femur, tibia, and fibula enclosed in a joint capsule. It contains an assortment of ligaments and tendons, which provide the structural framework for the joint. The knee permits flexion and extension as well as slight internal and external rotation of the lower leg.

The knee is at high risk for injury when exposed to force, especially in individuals who participate in athletics and other vigorous activities. A knee injury can be painful and life-altering, but with the right kind of intervention, pain and suffering can be relieved and normal function can be restored.

Methods of reducing and treating knee injuries have changed with the development and improvement of knee braces over time. A significant problem, yet to be addressed in the current design of knee braces created for athletes, are the number of players that experience performance limitations when competing while braced. In football, for example, skill players like receivers, kickers, and running backs report that current knee braces limit both their speed and agility. Offensive and defensive linemen, who are at greatest risk for knee ligament injuries, have found knee bracing to be beneficial but also report performance limitations.

Most current knee brace frames incorporate standard bilateral, polycentric hinges which provide a stabilizing force to the flexion and extension of the knee. While this method attempts to mimic the joint's natural movement, it falls short in allowing the knee the ability to internally and externally rotate. During the last 30° of extension, the tibia or femur must externally or internally rotate, approximately about 10°. While this rotation may seem small, it remains essential for healthy motion of the knee and maximal performance. Rotation must occur to achieve full flexion and extension. If the tibia or femur is held in a fixed position, this can alter the mechanics of the knee and lead to increased chance of injury.

By eliminating the joint's ability to rotate, the brace begins to fight against the joint's true motion, decreasing the ability of the athlete to move with fluidity and increasing the risk of brace migration and injury.

### BRIEF DRAWING DESCRIPTIONS

FIG. 1 illustrates an anterior view of the Performance Knee Orthosis.

FIG. 2 illustrates a posterior view of the Performance Knee Orthosis.

FIG. 3 illustrates a medial view of the Performance Knee Orthosis highlighting the fastening system.

FIG. 4 illustrates a lateral view of the Performance Knee Orthosis highlighting the cable tensioning system incorporated into the fastening system.

FIG. 5 illustrates a lateral view of the Performance Knee Orthosis on the leg of a wearer.

FIG. 6 illustrates a medial view of the Performance Knee Orthosis on the leg of a wearer.

FIG. 7 Illustrates a lateral view of the Performance Knee Orthosis in a position of flexion.

FIG. 8 Illustrates a perspective view of the hinge system in a position of flexion.

FIG. 9 Illustrates an exploded view of the hinge system specifically illustrating the uniaxial joint and multiaxial joints.

FIG. 10 Illustrates an exploded view of the multiaxial joint highlighting the connection between the rotational ball connector and the ball receiver.

FIG. 11 Illustrates an exploded view of the uniaxial joint highlighting the connection between the uniaxial joint and the receiving lid.

FIG. 12 Illustrates a perspective view of the hinge system highlighting the connection between the rotational ball connector and the ball receiver as well as the connection between the uniaxial receiver and the uniaxial connector.

FIG. 13 Illustrates a side view of the hinge system integrated into the upper and lower brace frame and the connection points of the uniaxial receiver and uniaxial connector and the rotational ball connector and the ball receiver.

FIG. 14 Illustrates a side view of the hinge system internally and externally rotating at the multiaxial joint.

FIG. 15A Illustrates a perspective view of the hinge system incorporating the slide mechanism, allowing for brace frame extension and compression.

FIG. 15B Illustrates a perspective view of the hinge system incorporating the t-slider, allowing for brace frame elongation and compression.

FIG. 15C Illustrates a perspective view of the hinge system incorporating a tubular rod system, allowing for brace frame elongation and compression and highlighting the movement of the slide mechanism screw.

FIG. 16 Illustrates a perspective view of the hinge system slide mechanism highlighting the movement of the receiving lid incorporating the slide mechanism on the receiving lid.

FIG. 17 Illustrates a perspective view of the hinge system incorporating the slide mechanism on the upper brace frame illustrating the movement of the upper brace frame on the elongated opening.

FIG. 18 Illustrates a perspective view of the hinge system incorporating a termination wedge that allows for customization of the allowed degrees of extension.

FIG. 19 Illustrates an exploded view of the cable tensioning system located on the brace frame.

### SUMMARY OF THE INVENTION

The embodiments herein disclose a device designed to be used by athletes at high risk for knee injury. This present invention provides prophylactic, post-injury, or postoperative support to the knee by limiting potentially harmful movements while the brace frame adjusts to the athlete's natural joint movement, reducing the severity, as well as the potential, of a future or recurring injury to the knee.

In one embodiment, the Performance Knee Orthosis includes a rigid, two-piece, asymmetrical, s-shaped brace frame that wraps around the anterior of the upper leg and the posterior of the lower leg. The two-piece brace frame consists of an upper brace frame and lower brace frame unified by a bilateral hinge system located on the medial and lateral side of the brace frame at the knee of the wearer. In one embodiment, the hinge system consists of a uniaxial joint, allowing for flexion and extension, a multiaxial joint, which allows for internal and external rotation, and a slide mechanism, which allows the brace frame to compress and elongate as needed for lateral movement of the leg.

The uniaxial joint consists of a uniaxial receiver, integrated into the hinge system, and a uniaxial connector, integrated into the upper brace frame. The multiaxial joint consists of a rotational ball connector, ball receiver, and termination peg. The rotational ball connector, integrated into the hinge system, locks into the ball receiver which is integrated into the lower brace frame. The ball receiver is located on the receiving lid and is integrated into the lower brace frame. The receiving lid encloses and secures the rotational ball connector to the ball receiver. A termination peg is attached to the receiving lid and restricts the allowable degrees of rotation to be limited to 10° or less. In another embodiment the termination peg can be attached to the receiving lid.

in one embodiment, a slide mechanism located at the connection point between the uniaxial joint and the upper brace frame. The slide mechanism consists of an elongated opening where a slide mechanism screw and slide mechanism nut are inserted and secured. The elongated opening allows the brace frame to extend and compress upon lateral movement of the wearer. This aids in antimigration of the brace frame on the leg of the wearer, a common cause of injury, as the hinge system performs a pistoning motion with the leg.

In another embodiment, a t-slider can serve as the slide mechanism. The t-slider, comprised of a vertical rod, a horizontal bar, and a t-stop, allows the brace frame to elongate and compress as the t-slider slides between the receiving lid and the t-stop.

In another embodiment, an alternative slide mechanism, such as a tubular rod system, could serve as the slide mechanism. The tubular rod system, located above the rotational ball connector on the multiaxial joint, connects to the receiving lid, and consists of a tube, rod, which allow for a controlled amount of brace frame elongation and compression.

Upper and lower termination walls, integrated into the posterior side of the upper brace frame and lower brace frame, in relation to uniaxial and multiaxial joints, prevent the brace from exceeding 180° of uniaxial extension.

In another embodiment, the uniaxial joint or multiaxial joint have an additional termination wedge on the upper-frame termination wall that allows for customization of the allowed degree of extension. A termination wedge, longer in width than the upper-frame termination wall, can be inserted into the upper-frame termination wall increasing the width. The increase in width lowers the degree of extension. This additional termination wedge can vary in width, allowing for varying degrees of extension.

The uniaxial joint, located near the condyle portion of the knee, multiaxial joint, located near the tibia, and slide mechanism work together to form the hinge system and provide a fully-integrated system that allows for up to 180° of flexion and extension, 10° of internal or external rotation, and up to 10 mm of frame elongation and compression.

In another embodiment, the hinge system includes a uniaxial joint, allowing for flexion and extension and a multiaxial joint, unified by a receiving lid, extending between the uniaxial and multiaxial joint.

In an additional embodiment, the hinge system can be used on any portion of the body that incurs a rotational force (i.e. the knee, ankle, wrist, hip, elbow, back, etc.)

In another embodiment, the rotational termination peg can be modified in height to allow for any desired degree of rotation from 1° to 45°.

The interior brace frame is lined with padding. The padding is removably, or in another embodiment, permanently, adhered to the brace frame. The exterior surface of the padding is lined with a gripping material, preventing brace migration. Condyle pads are removably adhered to the interior of the hinge system providing additional padding to the condyles of the knee.

The frame also houses a fastening system for securing the frame to the leg of the wearer. The fastening system is comprised of multiple fasteners as well as a cable tensioning system. The fasteners are integrated into the lower brace frame and upper brace frame through a series of fastener connection rings and integrated fastener slots. Each fastener is lined with hook and loop material and attach to themselves, securing the brace to the wearer. A rubber or silicone grip tip is placed near the end of each fastener for ease in grip and fastener adjustment.

A cable tensioning system located on the exterior of the brace frame, or in another embodiment, on the individual fastener, consist of adjustment dials, tensioning cables, a cable tunneling system, and cable guides. The cable tensioning system is used to create a customized, and more secure, fastener fit, which prevents migration of the brace frame and allows for sequential, dynamic, and gradual adjustment of the fastening system. The dials, consisting of a dial head, spool, cable tensioning system, and a release mechanism, located on the lateral portion of the brace frame or the fastener, are easily accessed by the user. Turning the dial clockwise creates tension and results in a tightening effect to the fastener. Pulling out on the dial releases the tension. In another embodiment the frame would attach to the wearer without the necessity of an fastener system.

Indications for this Performance Knee Orthosis include: prophylactic knee protection, tendonitis, ligament tears, meniscal injuries, arthritis, muscle sprains, and muscle strains, among others.

### DETAILED DESCRIPTION OF THE DRAWINGS

Embodiments are described more fully below with reference to the accompanying figures which illustrate specific embodiments. These embodiments are disclosed in sufficient detail to enable those skilled in the art to practice the invention. However, embodiments may be implemented in many different forms and should not be construed as being limited to the embodiments set forth. The following detailed description is, therefore, not to be taken in a limiting sense in that the scope of the present invention is defined by the claims.

The embodiments herein disclose a device designed to be used by athletes at high risk for knee injury. This present invention provides prophylactic, post-injury, or postoperative support to the knee by limiting potentially harmful movements while the frame adjusts to the athlete's natural joint movement, reducing the severity, as well as the potential, of a future or recurring injury to the knee.

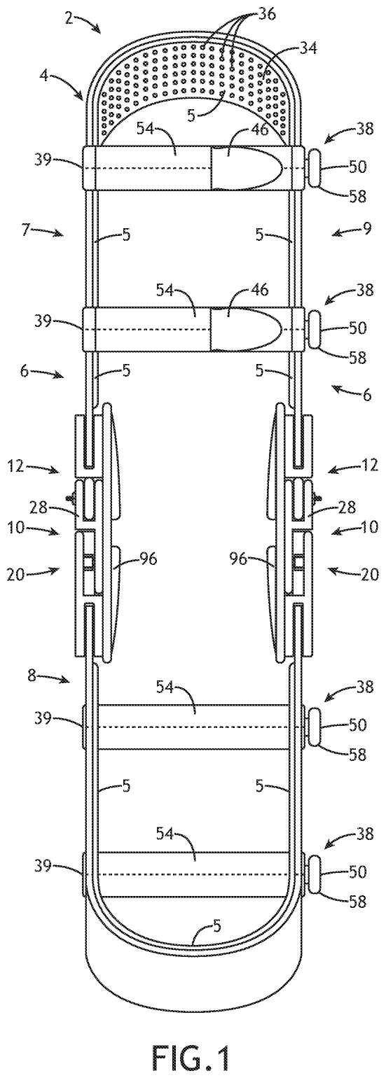

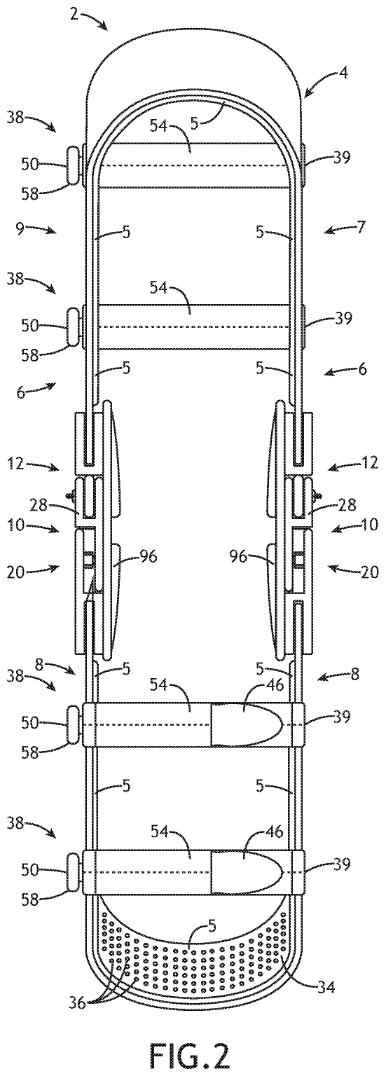

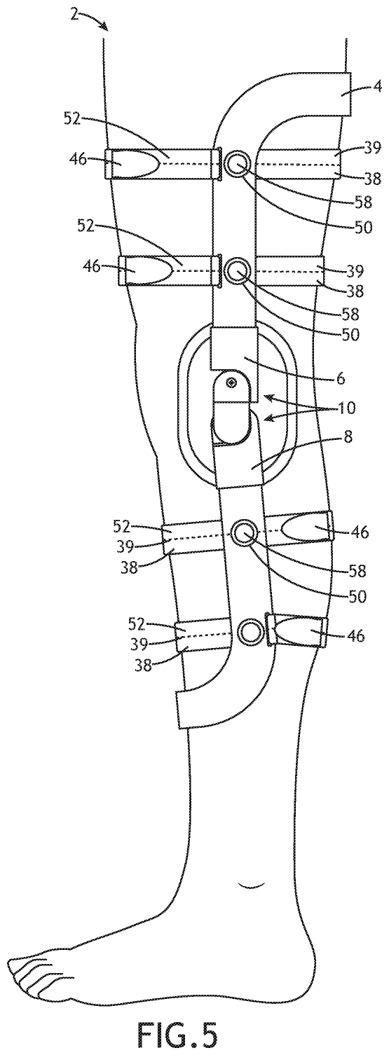

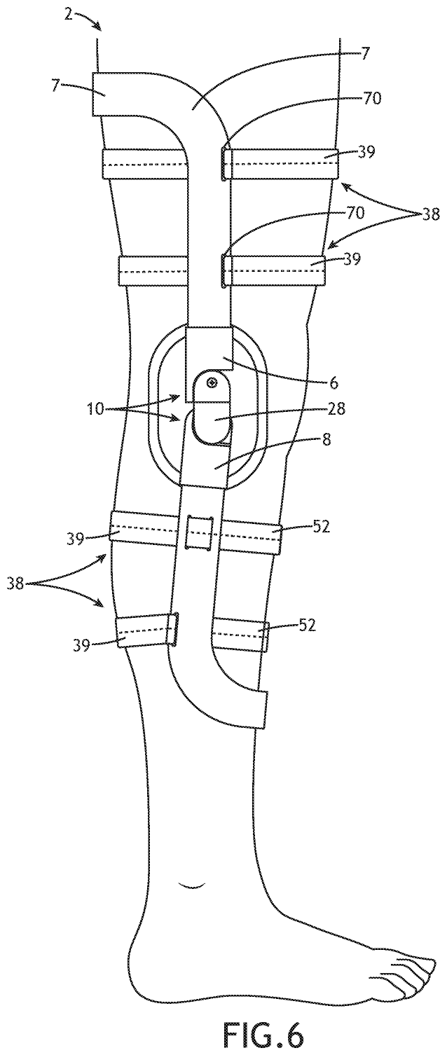

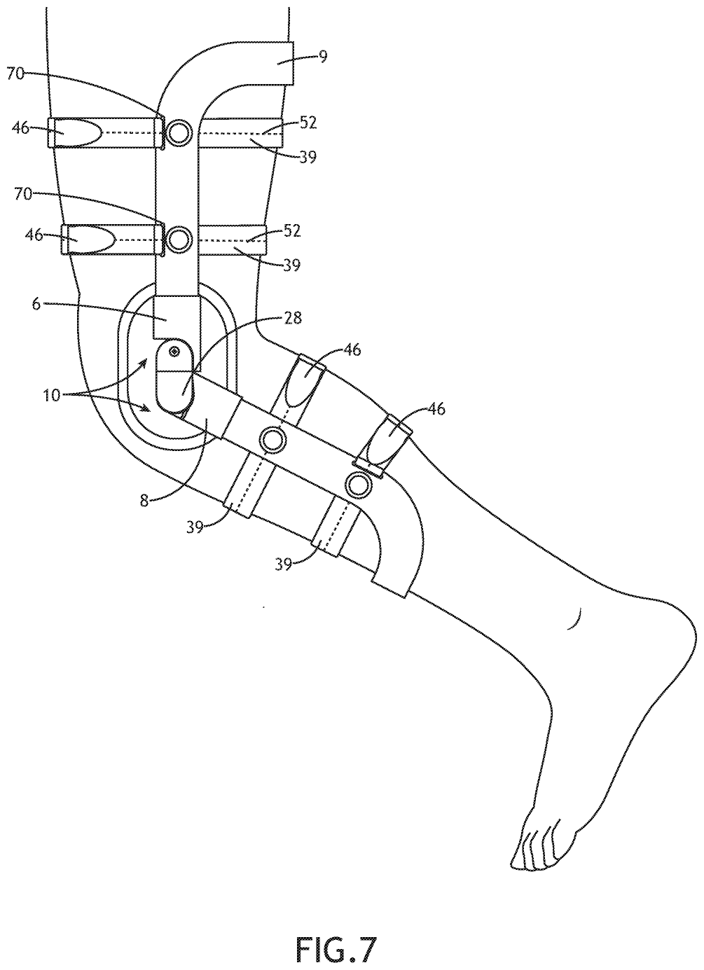

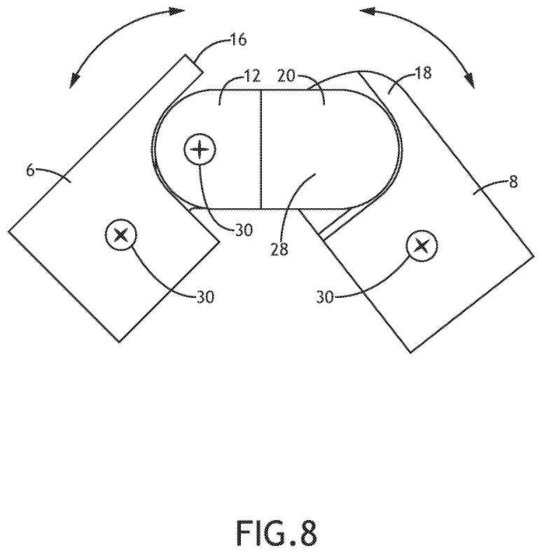

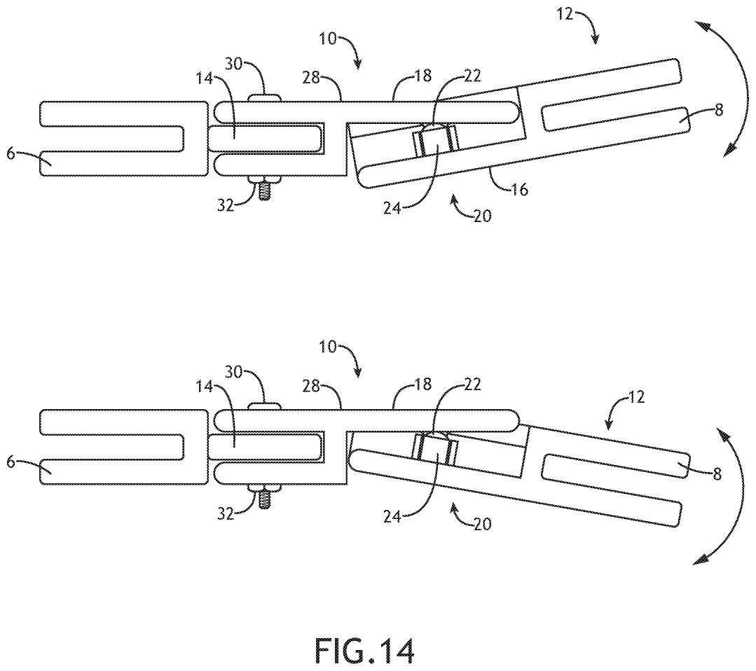

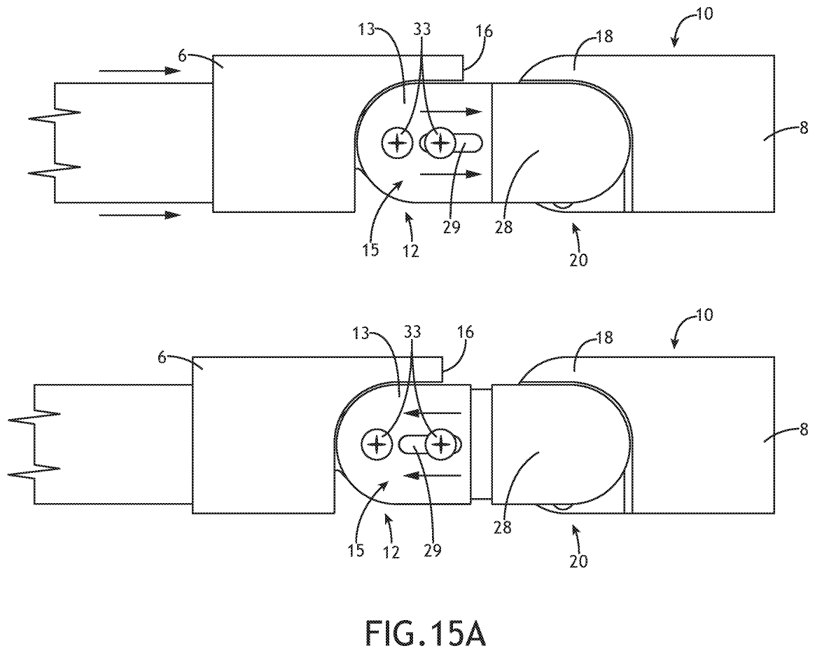

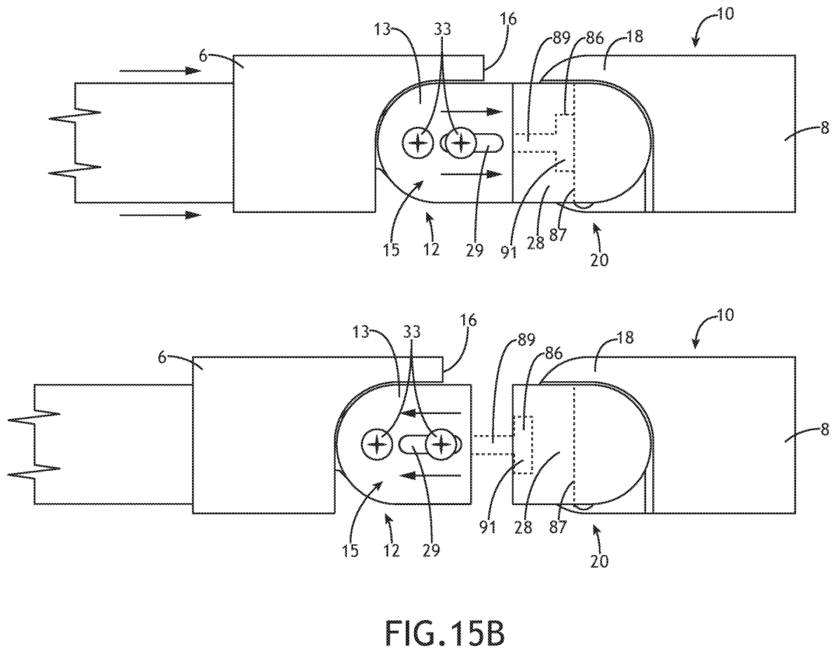

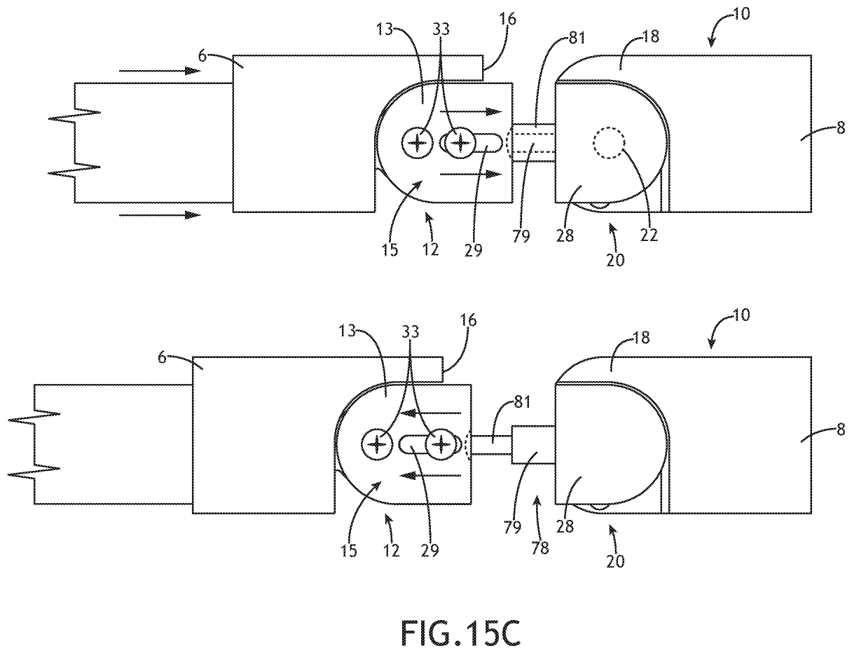

In one embodiment, the Performance Knee Orthosis 2 includes a rigid, two-piece, asymmetrical, s-shaped brace frame 4 that wraps around the anterior of the upper leg and posterior of the lower leg of the wearer as shown in FIG. 5. In additional embodiments, the brace frame 4 can be made of titanium, aluminum, or another lightweight metal. The two-piece brace frame 4 consists of an upper brace frame 6 and lower brace frame 8, unified by a hinge system 10 located on the medial side 7 and lateral side 9 of the brace frame 4, as shown in FIG. 1, FIG. 2, FIG. 5, and FIG. 6. In one embodiment, the a hinge system 10 consists of a uniaxial joint 12, allowing for flexion and extension as shown in FIG. 7 and FIG. 8, a multiaxial joint 20, which allows for internal and external rotation as shown in FIG. 14, and a slide mechanism 15 which allows the brace to compress and elongate as needed for lateral movement of the leg, as shown in FIG. 15A.

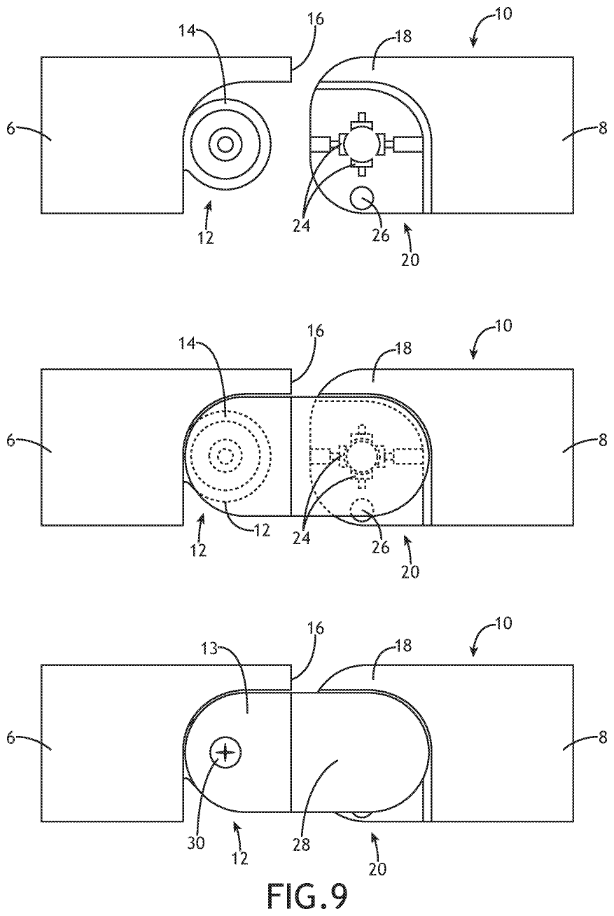

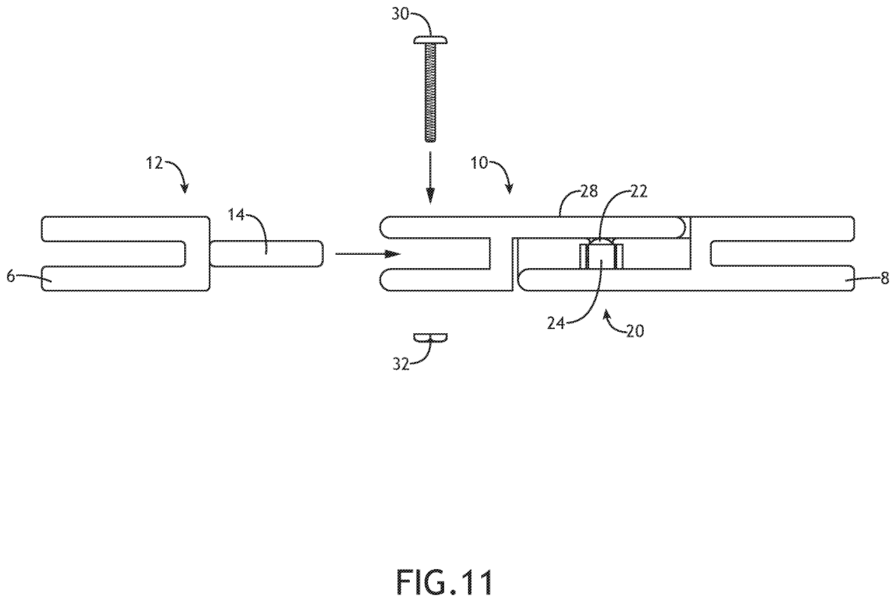

The uniaxial joint 12 consists of a uniaxial connector 14, a receiving lid 28, a screw opening 31, a screw 30, and nut 32 collectively permanently adhered to the upper frame 6 as shown in FIG. 9 and FIG. 11. In one embodiment, the uniaxial joint 12 can be made of a ceramic metal alloy or a thermoplastic polymer.

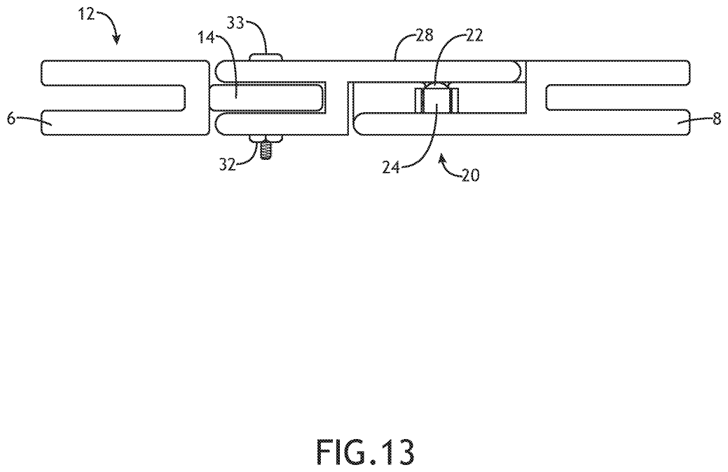

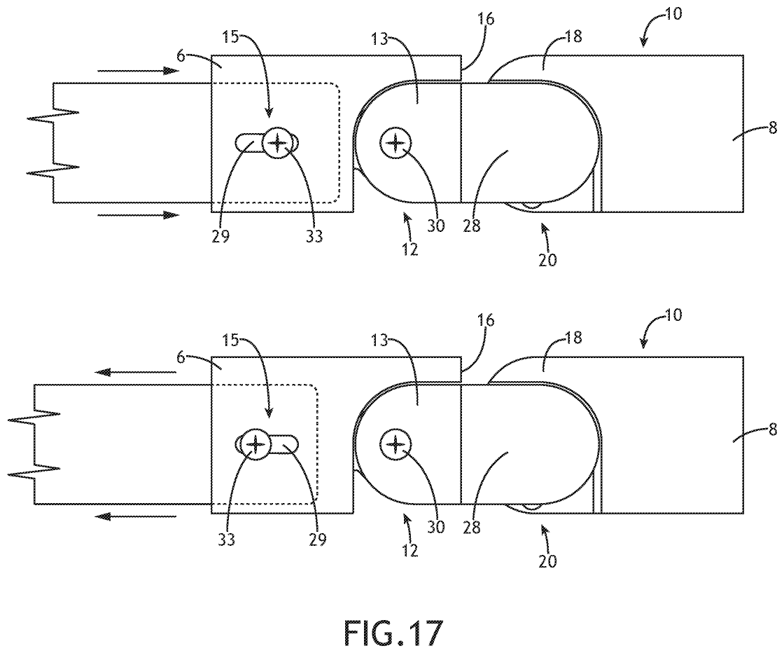

In one embodiment, a slide mechanism 15 is located at the connection point between the uniaxial joint 12 and the upper frame 6. The slide mechanism 15 consists of an elongated opening 29 where a slide mechanism screw 33 and slide mechanism nut 37 are inserted and secured as shown in FIG. 13. The elongated opening 29 allows the brace frame 4 to extend and compress along the length of the elongated opening 29 and upon activation of the wearer as shown in FIG. 17. This aids in antimigration of the brace frame 4 as the hinge system 10 can perform a pistoning motion with the body as shown in FIG. 15A.

In another embodiment, an alternative slide mechanism 15, such as a t-slider 86, comprised of a vertical rod 89, a horizontal bar 91, and a t-stop 87, allows the brace frame 4 to shorten or lengthen as the t-slider 86 slides between the receiving lid 28 and the t-stop 87 as shown in FIG. 15B.

In another embodiment, an alternative slide mechanism, such as a tubular rod system 78 would replace the slide mechanism 15 as shown in FIG. 15C. The tubular rod system 78, located above the rotational ball connector 22, connects to the ball receiving lid 28, and consists of a tube 79 and rod 81, which allow for a controlled elongation and compression of the brace frame 4. The tubular rod 81 connects to the multiaxial joint 20 as shown in FIG. 15C.

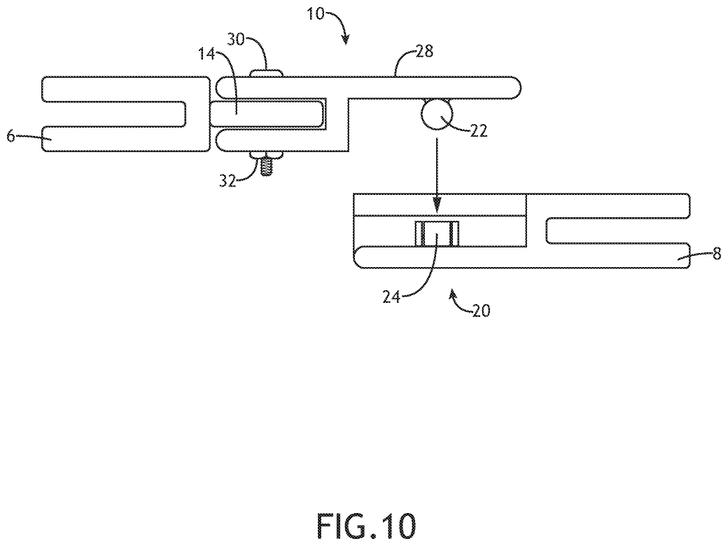

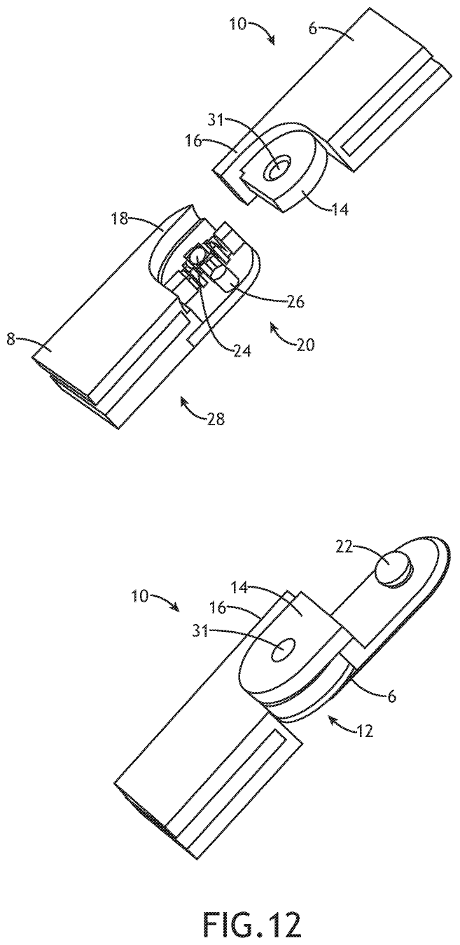

In one embodiment, the multiaxial joint 20 consists of a ball receiver 24, and termination peg 26, as shown in FIG. 9, and a rotational ball connector 22. The rotational ball connector 22 is integrated into the upper brace frame 6 and locks into the ball receiver 24. The ball receiver 24 is located on the receiving lid 28 and is integrated into the lower brace frame 8 as shown in FIG. 10. The receiving lid 28 encloses and secures the rotational ball connector 22 to the ball receiver 24. In one embodiment, the ball receiver 24 is made of a high-density polyethylene or another thermoplastic polymer and the rotational ball connector 22 is made of a polytetrafluoroethene coated metal with a low coefficient of friction. A termination peg 26 is attached to the receiving lid 28 which houses the ball receiver 24 and attaches to the rotational ball connector 22 as shown in FIG. 12 and restricts the allowable degrees of rotation to be limited 10° or less.

An upper-frame termination wall 16 and lower-frame termination wall 18, integrated into the posterior side of the upper brace frame 6 and lower brace frame 8 in relation to uniaxial joint 12 and multiaxial joint 20 as shown in FIG. 8 prevent the brace frame 4 from exceeding 180° of uniaxial extension.

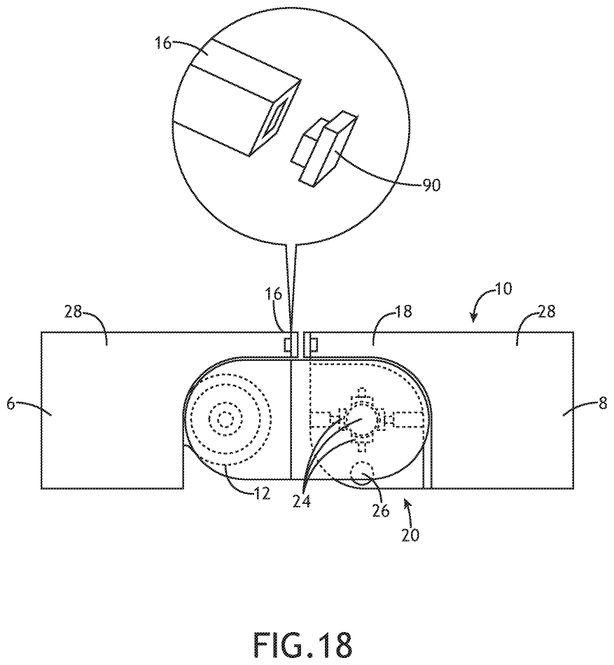

In another embodiment, the uniaxial joint 12 or multiaxial joint 20 has a termination wedge 90 on the upper-frame termination wall 16 that allows for customization of the allowed extension as shown in FIG. 18. A termination wedge 90 wider than the upper-termination wall 16 can be inserted into the upper termination wall 16 increasing the width of the upper-termination wall 16. The increase in width lowers the degrees of extension allowed around the multiaxial joint 20 and uniaxial joint 12. This additional termination wedge 90 can vary in width, allowing for varying degrees of extension.

In another embodiment, the hinge system 10 includes a uniaxial joint 12, allowing for flexion and extension and a multiaxial joint 20 unified by a receiving lid 28 extending between the uniaxial and multiaxial joint as shown in FIG. 8.

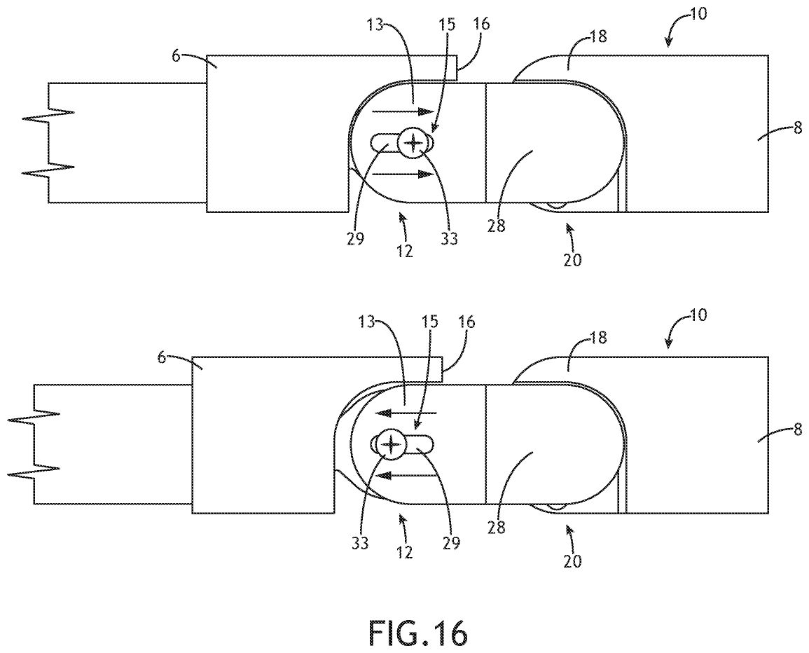

The uniaxial joint 12 located near the condyle portion of the knee, the multiaxial joint 20 located near the tibia, and a slide mechanism 15 work together to form the hinge system 10 and provide a fully integrated system as shown in FIG. 15A-C, allow for 180° of flexion and extension, 10° of internal or external rotation, and a small amount of elongation as shown in FIG. 16.

In an additional embodiment, the hinge system 10 can be used on any portion of the body that incurs a rotational force (i.e. the knee, ankle, wrist, hip, elbow, back, etc.)

The termination peg 26 as shown in FIG. 9 can be modified in height to allow for any desired degree of rotation from 1° to 45°.

The interior brace frame 5 is lined with padding 34 in FIG. 1. In one embodiment the padding is made of foam or the like. The padding 34 is removably, or in another embodiment, permanently, adhered to the brace frame 4. The padding 34 is lined with a gripping material 36 as shown in FIG. 2. In one embodiment, the gripping material 36 is made of rubber or silicone which interacts with the skin of the wearer, reducing brace frame 4 migration. Condyle pads 96, as shown in FIG. 1, are removably adhered to the interior of the hinge system 10 providing additional padding to the condyle of the knee.

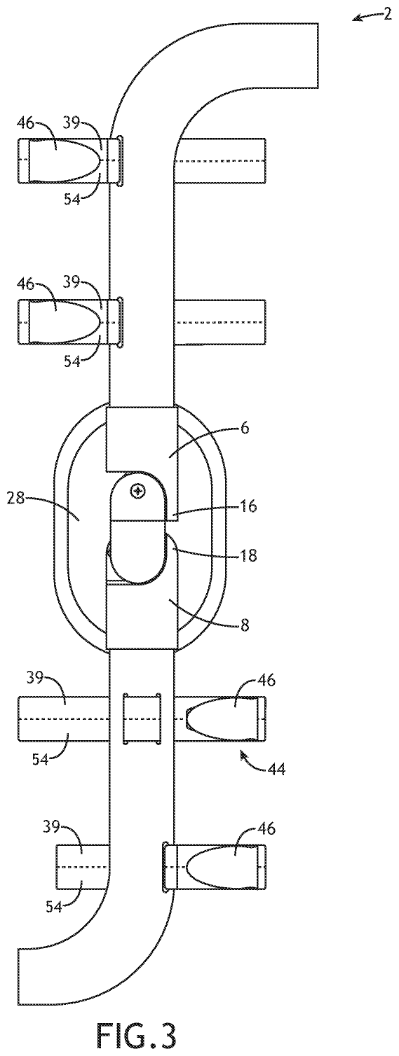

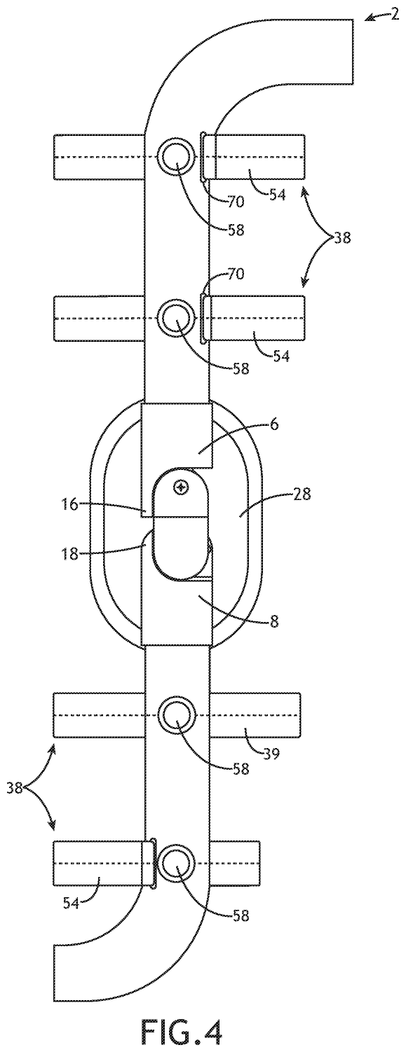

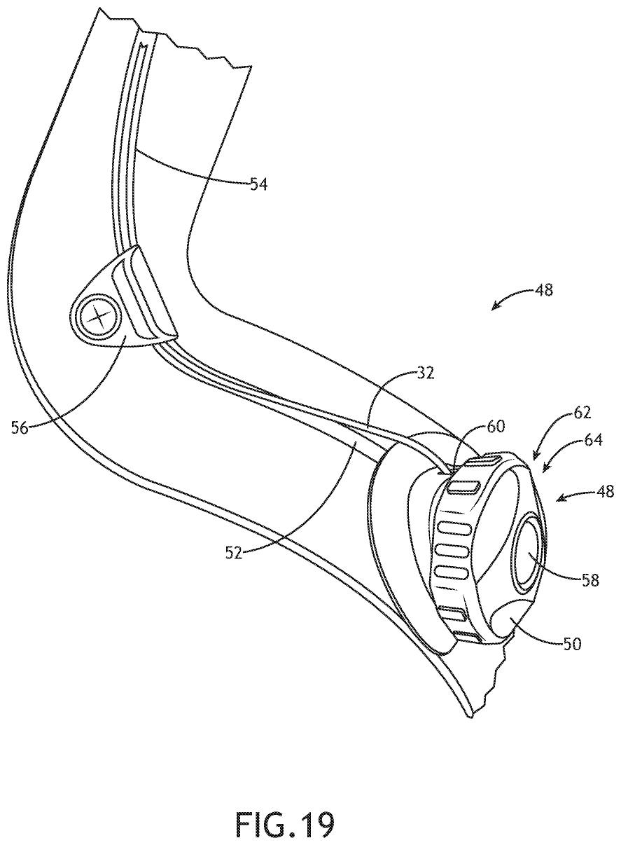

The brace frame 4 also houses a fastening system 38, as shown in FIGS. 5 and 6, for securing the frame 4 to the leg, comprised of individual fasteners 39 as well as a cable tensioning system 48, as shown in FIG. 19. In one embodiment, the fasteners 39 are located on the upper leg and lower leg and lined with hook and loop material 44 and attach to themselves, securing the brace frame 4 to the wearer. A rubber or silicone grip tip 46 is placed near the end of each fastener 39 for ease in finger gripping and fastener adjustment as shown in FIG. 3. The fastening system 38 is integrated into the brace frame 4 through a series of integrated slots 70 as shown in FIG. 4 and in another embodiment rings.

The cable tensioning system 48 located on the exterior of the brace frame 4, or in another embodiment on the fastening system 38 consisting of adjustment dials 50, a tensioning cable 52, a cable tunneling system 54, and cable guides 56, as shown in FIG. 19, are used to create a customized fit which allows for sequential, dynamic, and gradual adjustment of the fastening system 38. The adjustment dials 50, consisting of a dial head 58, spool 60, cable tensioning system 62, and a release mechanism 64, located on the lateral side of the brace frame 9, is easily accessed by the wearer. Turning the dial 50 clockwise creates tension and results in a tightening effect to the fastening system 38 as shown in FIG. 19. This embodiment allows multiple fasteners to be adjusted at the same time and in unison.

In another embodiment, the cable tensioning system 48 is located on the individual fasteners 39 of the fastening system 38. This embodiment allows an individual fastener 39 to be adjusted individually as shown in FIG. 2.

Indications for this Performance Knee Orthosis include: tendonitis, ligament tears, meniscal injuries, arthritis, muscle sprains, and muscle strains, among others.

1. We claim a device for supporting the knee, comprising:
a frame,
a hinge system allowing for flexion and extension and internal and external rotation wherein said hinge system attaches to said frame, and
a fastener system, integrated into said frame.

2. We claim a device for supporting the knee, comprising:
a frame,
a hinge system allowing for flexion and extension and internal and external rotation, wherein said hinge system attaches to said frame, and
a fastener system, integrated into said frame.

3. We claim a device for supporting the knee, comprising:
a frame,
a hinge system comprising a uniaxial joint and a multiaxial joint wherein said hinge system attaches to said frame, and
a fastener system, integrated into said frame.

4. We claim a device for supporting the knee, comprising:
a frame,
a hinge system comprising a uniaxial joint, multiaxial joint, a slide mechanism, and termination members, wherein said hinge system attaches to said frame, and
a fastener system, integrated into said frame.

5. We claim a device for supporting the knee, comprising:
a frame,
a hinge system, consisting of a uniaxial joint and a multiaxial joint, where said uniaxial joint consists of a uniaxial connector and a uniaxial receiver, and said multiaxial joint consists of a rotational ball connector and a ball receiver, wherein said hinge system attaches to said frame, and
a fastener system, integrated into said frame.

6. We claim a device for supporting the knee, comprising:
a frame,
a hinge system, consisting of a uniaxial joint, multiaxial joint, wherein said uniaxial joint consists of a uniaxial connector and uniaxial receiver, wherein said multiaxial joint consists of a rotational ball connector, a ball receiver, a termination peg, an upper frame termination wall, and a lower frame termination wall, wherein said hinge system attaches to said frame,
a fastener system integrated into said brace frame through a series of strap slots and strap connection rings comprising multiple fasteners and a cable tensioning system, and
padding adhered to the interior of said brace frame.

7. We claim a device of claim 6 wherein said frame is made of titanium, carbon fiber, aluminum, or another lightweight metal.

8. We claim a device of claim 6 wherein said ball receiver is made of high-density polyethylene or another thermoplastic polymer.

9. We claim a device of claim 6 wherein said uniaxial joint is made of a ceramic metal alloy, carbon fiber, or a thermoplastic polymer.

10. We claim a device of claim 6 wherein said rotational ball connector is made of titanium, carbon fiber, or a polytetrafluoroethene coated metal with a low coefficient of friction.

11. We claim a device of claim 6 wherein said cable tensioning system is located on the individual fasteners of said fastener system.

12. We claim a device of claim 6 wherein said cable tensioning system is located on said brace frame.

13. We claim a device of claim 6 wherein said cable tensioning system consists of adjustment dials, a tensioning cable, a cable tunneling system, and cable guides.

14. We claim a device of claim 13 wherein said dials of said cable tensioning system, consists of a dial head, spool, cable tensioning system, and a release mechanism.

15. We claim a device of claim 6 where said fastener system includes strap slots and connection rings.

16. We claim a device of claim 6 where said fastener system includes grip tips.

17. We claim a device of claim 16 where said grip tips are made of rubber or silicone or another flexible material.

18. We claim a device of claim 6 where said padding is removably or permanently adhered to said frame.

19. We claim a device of claim 18 where said padding is lined with a gripping material.

20. We claim a device of claim 19 where said gripping material is made of rubber or silicone.

21. We claim a device for supporting the knee, comprising:
a frame,
a hinge system, consisting of a uniaxial joint, multiaxial joint, wherein said uniaxial joint consists of a uniaxial connector and uniaxial receiver, wherein said multiaxial joint consists of a rotational ball connector, a ball receiver, a termination peg, an upper frame termination wall, and a lower frame termination wall, wherein said hinge system attaches to said frame,
a fastener system integrated into said brace frame through a series of strap slots and strap connection rings comprising multiple fasteners and a cable tensioning system, and
padding adhered to the interior of said brace frame.

22. We claim a device for supporting the knee, comprising:
a frame,
a hinge system, consisting of a uniaxial joint, multiaxial joint, where said uniaxial joint consists of a uniaxial connector and uniaxial receiver, where said multiaxial joint consists of a rotational ball connector, a ball receiver, a termination peg, a slide mechanism, an upper frame termination wall, and a lower frame termination wall, wherein said hinge system attaches to said frame,
a fastener system integrated into said brace frame through a series of strap slots and strap connection rings comprising multiple fasteners and a cable tensioning system, integrated into said frame, and
padding adhered to the interior of said brace frame.

23. We claim a device of claim 22 wherein said slide mechanism consists of an elongated opening.

24. We claim a device of claim 22 wherein said slide mechanism consists of a vertical rod, horizontal bar, and t-stop, an upper frame termination wall and lower frame termination wall.

26. We claim a device of claim 22 wherein said slide mechanism consists of a tube and a rod and connects to said multiaxial joint termination members.

27. We claim a method for supporting the knee, comprising:
a frame,
a hinge system allowing for controlled flexion and extension and controlled internal and external rotation of the knee, wherein said hinge system attaches to said frame, and
a fastener system, is integrated into said frame and attaches to the leg of the wearer.

28. We claim a method of claim 27 which further comprises a termination wedge to further limit extension.

29. We claim a method of claim 27 wherein said frame, said hinge system, and said fastener system can be used to support another anatomical portion of the body that incurs a rotational force.
